(19) United States
(12) Patent Application Publication
(10) Pub. No.: US 2015/0136341 A1
(43) Pub. Date: May 21, 2015

(54) NEONATAL PRIVACY SCREEN

(71) Applicant: Nexxspan Healthcare, LLC, Lithia, FL (US)

(72) Inventor: Alexander Bally, Marston Mills, MA (US)

(21) Appl. No.: 14/541,957

(22) Filed: Nov. 14, 2014

Related U.S. Application Data
(60) Provisional application No. 61/904,792, filed on Nov. 15, 2013.

(51) Int. Cl. A47G 5/00

(52) U.S. Cl. CPC A47G 5/00; A61B 2217/002
USPC 160/368.1; 29/428; 29/426.2

(57) ABSTRACT

Disclosed is a privacy screen assembly comprising: a screen; a support having a first end and a second end, the screen attached to the support proximate to the first end; and a mount assembly, the mount assembly attached to the support proximate to the second end, the support offsetting the screen from the mount assembly.

### REFERENCE TO RELATED APPLICATIONS

This application claims priority to U.S. Provisional Application No. 61/904,792, filed Nov. 15, 2013, which is hereby specifically incorporated by reference herein in its entirety.

### TECHNICAL FIELD

This disclosure relates to a privacy screen assembly. More specifically, this disclosure relates to a privacy screen assembly that, in various embodiments, is adjustable and allows for improved performance in tight quarters such as in a hospital room or in a neonatal intensive care unit (“NICU”).

### BACKGROUND

Throughout hospitals and extended care facilities, bed and examination areas are often provided privacy through the use of ceiling-mounted movable fabric curtains. There are many manufacturers of these products and all are essentially the same basic idea with limited variation. Typically, a track is provided which consists of an extruded aluminum channel. The track is attached to a conventional or dropped ceiling, and traces a path around the area to be sequestered. A series of rollers slide within that track, each of the rollers having a chain hanging from it. The chains, in turn, each end in a hook. To drape a curtain around the examination area, each hook is attached to a grommet in the curtain, a series of grommets being provided along the curtain's upper edge. Once the hooks have been attached to the curtain's grommets, the curtain hangs from the ceiling and can slide along the track's path. Usually, this path is arc-shaped, extending around three sides of a bed or examination area (the fourth side often being a wall). A health care professional such as a doctor or nurse pulls the curtain to slide it along the track, to open and close a space around the bed.

Because of the track, the rollers, and the moving parts, the noise of the curtain sliding along the aluminum track is very disturbing to patients. Particularly in a hospital environment, patients need their rest. Yet, the movement of the curtain along the track not only disturbs the patient in the bed surrounded by the curtain, but also the patient in the adjoining bed (which is a problem in general, and particularly so if that second patient was sleeping). Maintenance, repair and laundering of these systems is highly inefficient.

Furthermore, the existing designs use a single entry and exit point for insertion or removal of the curtain at an end of the track (or at the two ends of the track at best). If one roller breaks in the sequence of rollers, all the rollers and the entire curtain up to that point need to be removed from the track so that the broken roller can be replaced. Likewise, the curtain can often disengage from the track. Particularly considering the large number of hooks and rollers and small parts, it is time consuming to maintain and repair the system and its parts, and to reattach the curtain. Yet the number of moving parts only increases the likelihood of breakage, the likelihood of the curtain disengaging from the track, the likelihood of the hooks disengaging from the grommets, and so forth.

The curtains themselves consist of a single, continuous, lengthy stretch of material (usually greater than 24 feet, and often 30 feet long) and are heavy as a result. Due to that length and size, they must be sent out to a costly commercial laundering facility. Moreover, the design of the system and the curtain's weight make it difficult to handle the curtain, with it being particularly cumbersome to hang or change it. One roller at a time must be inserted into the track and then one hook at a time must be attached to the heavy curtain. As there are approximately two grommets per linear foot, the mounting or removal of the curtain (whether for maintenance, repair, cleaning, or replacement purposes) is a considerable chore that usually requires the efforts of two maintenance workers.

The utility and aesthetics of the system are also severely lacking. To pass beyond the curtain into the bed area, the health care professional must pull the curtain from the far end—i.e. he or she cannot simply enter in the middle. Also, the movement of the curtain is often not smooth or stable along the track, and portions of the curtain can jam up when the moving parts interfere with each other. The curtain can also get tangled as the hooks catch each other and/or sections of the curtain fabric. The use of the chains, rollers and hooks also results in a configuration where the curtain hangs a significant distance below the track, leaving a relatively large gap through which unwanted light can enter. In terms of both appearance and space, the existing curtains stack or gather together in an extremely wide silhouette—for example, 24 feet of curtain stack as approximately 4 feet, in other words, as a width of fabric approximately 4 feet wide. Moreover, since the curtain is a single continuous length, it can only be stacked together in one bundle. For all practical purposes, curtains in that system cannot be layered one on top of the other even if desired.

Further, in neonatal ICU units, mothers spend a lot of time with their babies for intensive skin-to-skin time. The more comfortable the environment can be made for the mother, the more time she will spend with the child. This includes breastfeeding. Some mothers are bashful and do not like to feed if there is a chance they will be seen. In these environments space is at a critical premium and large traditional curtains cannot be implemented. As a result, there is a need for a “privacy curtain” that is not a barrier against nurse supervision and yet provides sufficient shielding to allow the mother to comfortably interact with her newborn.

### SUMMARY

Disclosed is a privacy screen assembly comprising: a screen; a support having a first end and a second end, the screen attached to the support proximate to the first end; and a mount assembly, the mount assembly attached to the support proximate to the second end, the support offsetting the screen from the mount assembly.

Also disclosed is a method of assembling a privacy screen assembly comprising: attaching a screen proximate to a first end of a support; and attaching the support to a mount assembly proximate to a second end of the support such that the support offsets the screen from the mount assembly.

Also disclosed is a method of using a screen assembly comprising: mounting the screen assembly onto a structure, the screen assembly including: a screen; a support having a first end and a second end, the screen attached to the support proximate to the first end; and a mount assembly, the mount assembly attached to the support proximate to the second end, the support offsetting the screen from the mount assembly; and moving the screen relative to the mount assembly.

Various implementations described in the present disclosure may include additional systems, methods, features, and advantages, which may not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures may be designated by matching reference characters for the sake of consistency and clarity.

FIG. 1 is a perspective view of a screen assembly according to a first embodiment of the present disclosure including a screen, a hanger, a hanger fitting, a curved support, and a mount assembly.

FIG. 2 is a perspective view of the mount assembly and curved support of the screen assembly of FIG. 1 attached to a rail which is attached to a post.

FIG. 3 is a cross-sectional view of an adapter and adapter addition of the mount assembly mounted on the rail.

FIG. 4 is a perspective view of the screen and curved support of the screen assembly of FIG. 1.

FIG. 5 is another perspective view of the screen and curved support of FIG. 4.

FIG. 6 is a front view of the hanger and a top of the screen of the screen assembly of FIG. 1.

FIG. 7 is a front view of the hanger and the top of the screen attached to the hanger fitting of the screen assembly of FIG. 1.

FIG. 8 is an enlarged front view of the hanger and the top of the screen attached to the hanger fitting with a side of the hanger fitting removed to show the hanger fitting engaging the hanger.

FIG. 9 is a perspective view of a screen assembly attached to a rail mounted on a wall.

FIG. 10 is a perspective view of a screen assembly attached to a rail mounted on a headwall.

FIG. 11 is a perspective view of a hanger with a screen and hanger fitting in accordance with another embodiment of the current disclosure.

FIG. 12 is a perspective view of the hanger and hanger fitting of FIG. 11 in an engaged orientation.

FIG. 13 is a perspective view of a hanger fitting in accordance with another embodiment of the current disclosure.

FIG. 14 is a perspective view of the hanger fitting of FIG. 13 engaged with the hanger and screen of FIG. 11.

FIG. 15 is a front view of a hanger fitting in accordance with another embodiment of the current disclosure in an unfolded orientation.

FIG. 16 is a perspective view of the hanger of FIG. 15 engaged with the hanger and screen of FIG. 11.

### DETAILED DESCRIPTION

The present disclosure relates generally to an adjustable privacy screen. More specifically, the present disclosure relates to a privacy screen that, in various embodiments, is supported from above and easily repositionable in a manner that allows for improved performance in tight quarters such as hospital rooms and more specifically neonatal intensive care units (NICU).

Disclosed is a privacy screen assembly and associated methods, systems, devices, and various apparatus. The screen assembly includes a screen, a hanger, a hanger fitting, a curved support, and a mount. It would be understood by one of skill in the art that the disclosed screen assembly is described in but a few exemplary embodiments among many. No particular terminology or description should be considered limiting on the disclosure or the scope of any claims issuing therefrom.

The present disclosure provides, in various embodiments, a very light weight “flag” or “screen” that is sized and positioned to just cover the desired area. The flag or screen orientation can be easily changed to the chosen direction and can be controlled and moved by the mother. It is designed, in various embodiments, not to be “architectural” or structural in any manner and is purposely “flimsy” in various embodiments so as not to clutter the NICU room any more than necessary.

In various embodiments, the flag or screen system is attached, at one end of a support, to a mount that engages a hospital rail system. This attachment point may allow the flag or screen system to be adjusted laterally along the rail as needed and eliminates mounting the structure to the wall. As a result, in various embodiments, no additional anchoring is needed. In various embodiments, the support is a rod. In various embodiments, the support further includes an arcuate portion such that the support is a curved support. In these embodiments, the curved support may be received into the mount in a manner that may allow the curved support to pivot relative to the mount. Furthermore, in various embodiments, a collar is provided that may be adjustable along the support to provide for height adjustment of the flag or screen system relative to the mount. This allows the support to reach over equipment and obstacles.

The opposing end of the curved support may terminate in a hanger fitting (also referred to as a hanger attachment) in various embodiments. The fitting may be any mechanism of engagement known in the art. The hanger fitting is configured to allow a hanger bar (also referred to as a hanger) to be removably attached thereto. Furthermore, the fitting is preferably pivotable about the end of the support such as the curved support, though the hanger attachment may not pivot about the end of the support in various embodiments.

In various embodiments, the hanger bar receives and supports the flag or screen. The flag or screen itself may have a top seam through which the hanger bar may be pushed or inserted. Similarly, the top of the flag or screen may be formed as a bolt or have a bolt cord installed therein such that the screen may be supported in a groove formed in the bottom of the hanger. In these embodiments, this allows the flag or screen to be easily removed and replaced as needed. In various embodiments, the overall structural arrangement allows the screen system to easily pivot, rotate, and swivel.

In various embodiments, the flag screen material is preferably a “disposable” non-woven fabrics such as, for example, TYVEK® so it can be easily changed and discarded when the baby leaves. If there is no risk of infection, the mother may take it home in various embodiments. Further, the screen material may be drawn on in various embodiments so that nurses and visitors, among others, can write and draw on the screen surface so it becomes personalized for the baby as a memento.

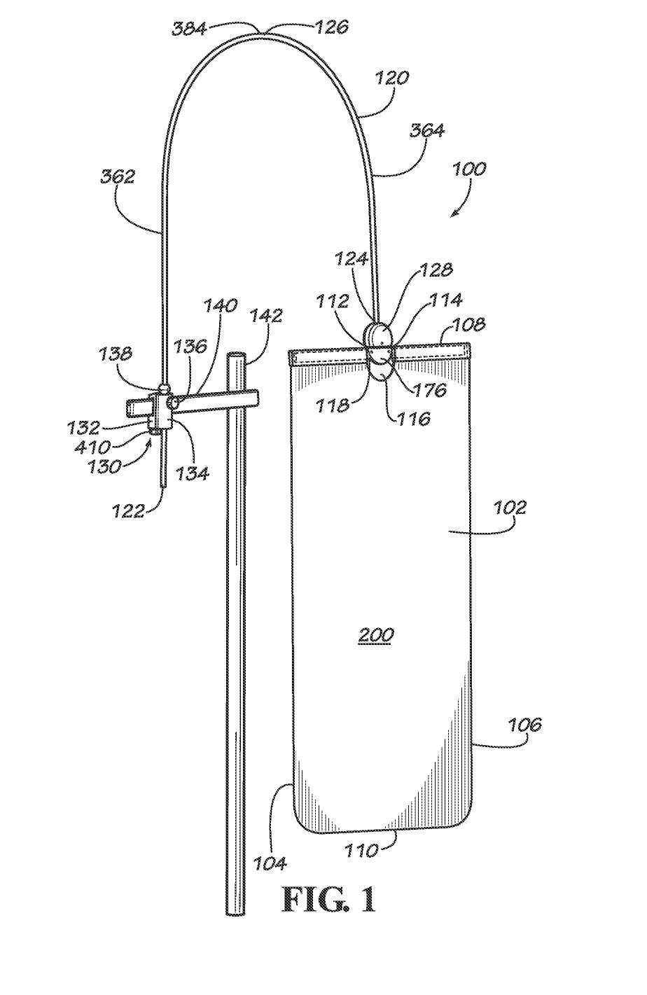

One embodiment of a privacy screen assembly 100 is disclosed and described in FIG. 1. As shown in FIG. 1, in various embodiments, the screen assembly 100 includes a screen 102, a hanger 118, a hanger attachment 128, a support 120, and a mount assembly 130. As shown in FIG. 1, in various embodiments, the screen assembly 100 is mounted onto a rail 140 which may be attached to a pole 142; however, as will be discussed below and with reference to other figures, in various other embodiments, the screen assembly 100 may be attached to other rails with configurations different than rail 140 or may be attached to another surface or structure other than a rail in various embodiments. In various embodiments, the screen assembly 100 is mounted on a wall, on a bed post, on a headwall, on a table, on a chair, on a desk or on other similar structures suitable for supporting the privacy screen assembly 100.

As shown in FIG. 1, the screen 102 includes a first side 104 and a second side 106 distal from the first side 104. In various embodiments, the distance from the first side 104 to the second side 106 may define a screen width. In the screen assembly 100, the screen 102 may also define a top end 108 and a lower end 110. In various embodiments, the screen 102 may define a first surface 200 extending between the first side 104, the second side 106, the top end 108, and the lower end 110. As shown in FIG. 1, in various embodiments, the top end 108 may define a screen opening 116 in the first surface 200 between a first opening end 112 and a second opening end 114 at the top end 108. As shown in FIG. 1, in various embodiments, the screen 102 may define the semi-circular-shaped opening 116; however, in various other embodiments, the shape of the screen opening 116 may have a square shape, an elliptical shape, or any other desired shape. Additionally, as shown in FIG. 1, in various embodiments, the corner between the first side 104 and the lower end 110 and the corner between the second side 106 and the lower end 110 defines rounded corners; however, in various other embodiments, the corners are not rounded. In various embodiments, the corners are chamfered or square. The disclosure of rounded, non-rounded, chamfered, or square corners, however, should not be considered limiting on the present disclosure.

The screen 102 may be constructed from flexible materials such as plastics, resins, textiles, and other similar materials that may be pleasing to the touch and also promote a safe medical environment. In various embodiments, the screen 102 may be constructed from nonwoven tough polyester. In particular, in various embodiments, the screen may be constructed from a nonwoven tough polyester such as the material sold by E.I. du Pont de Nemours and Company (DuPont), Wilmington, Del. under the trade name TYVEK®, or any other similar material. In various embodiments, the material chosen for the screen 102 may be a material that a user can write on. In various embodiments, materials such as felt tipped pens, markers, or other similar materials may be used by a user to write on the screen. In various embodiments, writing on the screen may serve utilitarian purposes such as recording medical considerations. In other embodiments, writing on the screen may serve aesthetic purposes such as a personalized screen with images and words of support. In various other embodiments, images, words, and other designs may be printed on the screen 102 prior to use.

As shown in FIG. 1, in various embodiments, the screen 102 may be mounted onto a hanger 118. In these embodiments, mounting the hanger 118 provides reinforcement for the top end 108 of the screen 102. However, in various other embodiments, the screen 102 may be reinforced through mechanisms including, but not limited to, fusing the screen 102 to the hanger 118, bonding the screen 102 to the hanger 118, or inserting the screen 102 in the hanger 118. In various embodiments, the hanger 118 is constructed from materials from the group including, but not limited to, various plastics, corrugated cardboard, metals, or any other material of sufficient strength to support a screen 102 but flexible and lightweight to reduce the weight supported by the support 120. In various embodiments, the hanger 118 and the screen 102 may be integrally formed using molding technology, ultrasonic welding, or other methods known in the art. In various other embodiments, the hanger 118 and screen 102 may be constructed from the same material. In various embodiments, the hanger 118 and screen 102 are constructed from recyclable materials described above.

In various embodiments, the hanger 118 may include a first side screen holder 176 and a second side screen holder 196 (shown in FIG. 5). In various embodiments, the first side screen holder 176 and the second side screen holder 196 may allow the hanger 118 to engage the hanger attachment 128.

In various embodiments, the support 120 may connect the screen 102 with the mount assembly 130. In various embodiments, the support 120 is a rod. The rod may be constructed from various plastics or metals such as stainless steel which are flexible but have strength to support the hanger 118 and screen 102. As shown in FIG. 1, in various embodiments, the support 120 may be a curved support 120. In various other embodiments, the support 120 may be a straight rod, a rectangular-shaped support, an angled support, or have other similar shapes. The disclosure of the shape of the support 120 should not be considered limiting on the present disclosure.

In various embodiments, the support 120 may include a first end 124 and a second end 122. As shown in FIG. 1, in various embodiments, the support 120 also includes a first vertical portion 364, a center portion 384, and a second vertical portion 362. As shown in FIG. 1, the first vertical portion 364 is defined as the portion of the support 120 between the center portion 384 and the first end 124 such that the first vertical portion 364 is proximate to the first end 124. The second vertical portion 362 is defined as the portion of the support 120 between the center portion 384 and the second end 122 such that the second vertical portion 362 is proximate to the second end 122. In the current embodiment, the second end 122 is oriented vertically below the first end 124 when the support 120 is in an upright position to hold the screen 102 higher, though in various other embodiments the second end 122 may be even with or above the first end 124. In the various embodiments the center portion 384 of the support 120 defines an arcuate portion 126 between the second end 122 and the first end 124. More specifically, in various embodiments, the arcuate portion 126 is defined between the first vertical portion 364 and the second vertical portion 362. In various embodiments, the support 120 may only include a single vertical portion, such as the first vertical portion 364, and the arcuate portion 126. In other embodiments, the support 120 may only include vertical portions connected by straight horizontal or angled portions and no arcuate portions are present. In other embodiments, various combinations of vertical portions and arcuate portions may be utilized as desired.

In various embodiments, the first end 124 of the support 120 may be connected to the hanger attachment 128. In various embodiments, when the support 120 is curved, the arcuate portion 126 defined in the support may enable the support 120 to rotate unobstructed relative to the mount assembly 130 because the arcuate portion 126 distances the first vertical portion 364 from the second vertical portion 362. In other words, the arcuate portion 126 offsets the first vertical portion 364 from the second vertical portion 362 and the mount assembly 130 and spaces apart the first vertical portion 364 from the second vertical portion 362. More specifically, in various embodiments, a person, structure, or support may be at a position below the arcuate portion 126 and between the first vertical portion 364 and the second vertical portion 362 without obstructing movement of the screen assembly 100. In these embodiments, the screen 102 is spaced apart from the mounting assembly 130 because of the support 120.

As shown in FIG. 1, in various embodiments, the screen assembly 100 also may include the mount assembly 130. In various embodiments, the mount assembly 130 may include an adapter 132, an adapter addition 134 attached to the adapter 132, a fastener knob 136, and a collar 138. In various embodiments, the mount assembly 130 may accept the first portion 362 of the support 120 through the adapter addition 134 such that the second end 122 of the support 120 is positioned below the mount assembly 130. In various embodiments, the collar 138 may enable the support 120 to be positioned at various heights by allowing different amounts of the support 120 to pass through the adapter addition 134. In various embodiments, the mount assembly 130 may enable the screen assembly 100 to be mounted onto the rail 140. In various embodiments, the mount assembly 130 may enable the screen assembly 100 to move slidably along the rail 140. In these embodiments, the fastening knob 136 may enable the mount assembly 130 to be locked into one position on the rail 140. As previously discussed, in various embodiments, the rail 140 may be mounted onto a post 142. In the present embodiment, the post 142 may be a 38 mm. post; however, in various other embodiments, the rail 140 may be mounted on a different type of post 142 or the rail 140 may not even be mounted on a post 142.

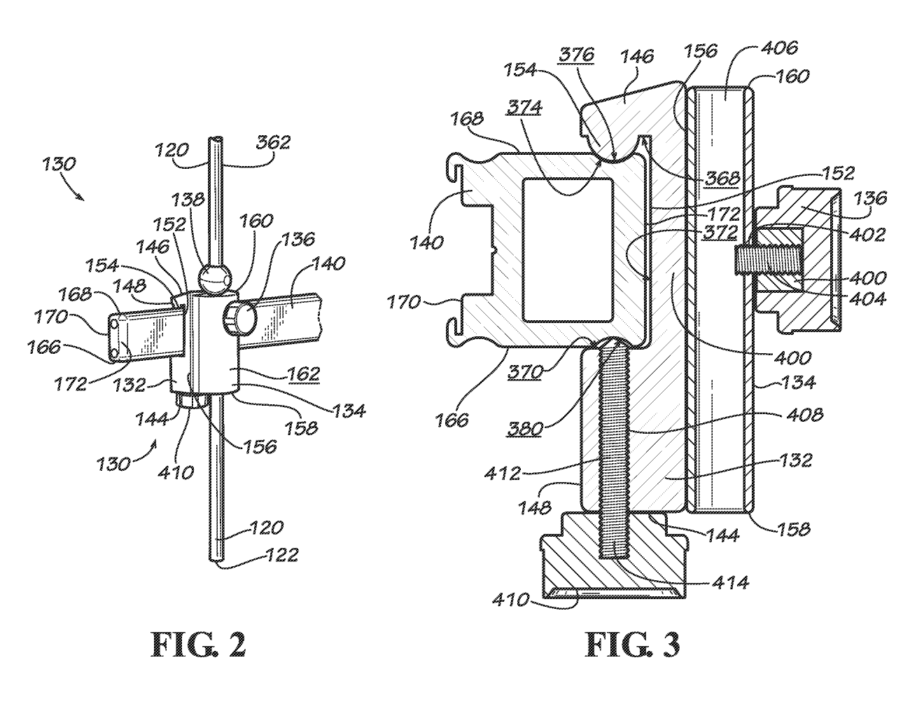

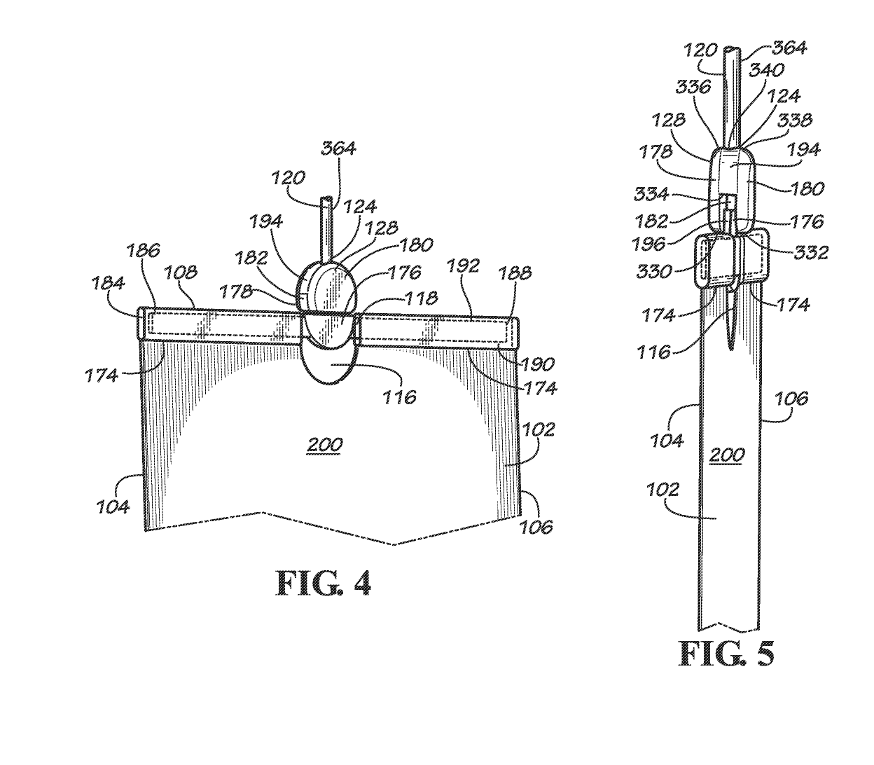

Focusing now on FIG. 2, the mount assembly 130 will be described in greater detail. As previously discussed, in various embodiments, the mount assembly 130 may include the adapter 132, the adapter addition 134, the fastening knob 136, securing knob 410, and the collar 138. The adapter 132 and rail 140 will be described in greater detail below with reference to FIG. 4. As shown in FIG. 2, in various embodiments, the adapter 132 may include a lower side 144 and a top side 146 distal from the lower side 144. The adapter 132 may also define a channel side 148 and an attachment side 156. In various embodiments, the channel side 148 defines a rail mating channel 152 between the top side 146 and the lower side 144. In various embodiments, the adapter 132 may define a rib 154 extending into the channel 152. As shown in FIG. 2, in various embodiments, the profile of the channel 152 enables the adapter 132 to be mounted onto the rail 140.

As shown in FIG. 2, in various embodiments, the adapter addition 134 may be attached to the attachment side 156 of the adapter 132. In various embodiments, the adapter addition 134 may be attached to the adapter 132 with glue, double sided tape, welding, screws, nuts and bolts, or any other suitable fastener, or the adapter addition 134 may be formed as a single unit with the adapter 132. Stated another way, in various embodiments, the adapter 132 and adapter addition 134 may be integrally formed as a single component.

The adapter addition 134 includes a lower side 158 and a top side 160 distal from the lower side 158. In various embodiments, the lower side 158 of the adapter addition 134 may be at the same end of the adapter 132 as the lower side 144 and the top side 160 of the adapter addition 134 may be at the same end of the adapter 132 as the top side 146. In various embodiments, the adapter addition 134 may define an outer surface 162 extending between the top side 160 and the lower side 158. In various embodiments, the adapter addition 134 may define a bore 406 (shown in FIG. 3) extending through the adapter addition 134 from the top side 160 to the lower side 158. In various embodiments, the bore 406 is sized to accept the support 120 and the support 120 may be inserted into the bore 406 of the adapter addition 134 such that the support 120 is partially enclosed by the adapter addition 134. In various embodiments, the bore 406 is sized to form a tight fit around the support 120 when the support is inserted; however, in various other embodiments, the bore 406 is sized to form a looser fit around the support 120. In various embodiments, sizing of the bore 406 may contribute towards the ease of moving and rotating the support 120 in the adapter addition 134. As shown in FIG. 2, in various embodiments, when the support 120 is inserted through the adapter addition 134, the second end 122 of the support 120 may be vertically below the mount assembly 130. In these embodiments, the mount assembly 130 is proximate to the second end 122 of the support 120 by attaching to the support 120 on the second vertical portion 362. Stated another way, the mount assembly 130 is attached to the support 120 nearer to the second end 122 of the support than the first end 124.

In various other embodiments, an adapter addition 134 may be omitted and the adapter 132 may define an opening through the adapter 132 from the top side 146 to the lower side 144. In various embodiments, the opening is sized to accept the support 120 and the support 120 may be inserted into the opening of the adapter 132 such that the support 120 is partially enclosed by the adapter 132. In various embodiments, the opening is sized to form a tight fit around the support 120 when the support is inserted; however, in various other embodiments, the opening is sized to form a looser fit around the support 120. In various embodiments, sizing of the opening may contribute towards the ease of positioning of the support 120 in the adapter 132.

In various embodiments, the support 120 may also be inserted through the collar 138. In various embodiments, the collar 138 has a spherical shape; however, in various other embodiments, the collar 138 may be cylindrical or cubed. The disclosure of spherical, cylindrical, and cubed collars, however, should not be considered limiting on the present disclosure, as the collar 138 may have any other desired shape. In various embodiments, the collar 138 may be constructed from material such as the material sold by E.I. du Pont de Nemours and Company (DuPont), Wilmington, Del. under the trade name DELRIN® or synthetic polymers such aliphatic polyamides. However the collar 138 may be constructed of any other material and the disclosure of DELRIN® and aliphatic polyamides should not be considered limiting on the current disclosure. In various other embodiments, the collar 138 may include other suitable mechanisms for adjusting height of the support 120. In various embodiments, the collar 138 may be movable along the support 120. In various embodiments, the collar 138 may be positioned at various locations along the support 120 to provide desired heights of the support 120. In these embodiments, the collar 138 may prevent the support 120 from passing through the adapter addition 134 at any portion of the support 120 above the collar 138. In these embodiments, the collar 138 may provide the ability to adjust the height of the support 120.

Although a collar 138 is described in the present embodiment, in various other embodiments, other height adjustment mechanisms may be utilized such as a bolt engaging the support 120, an O-ring positioned on the support 120 above the mount assembly 130, a silicone tube positioned on the support 120 above the mount assembly 130, or a pin locking mechanism with a pin that engages various openings defined in the support 120 at various heights. In various embodiments, the mount assembly 130 may directly attach to the support 120; however, in various other embodiments, the mount assembly may indirectly attach to the support 120. For example, in various embodiments, a pivotable arm, a hook, or a pin may connect the support 120 to the mount assembly 130; however, in other embodiments, other mechanisms for indirectly attaching the support 120 to the mount assembly 130 may be utilized.

As shown in FIG. 2, in various embodiments, the mounting assembly 130 may also include a fastening knob 136. In various embodiments, the fastening knob 136 includes a fastener 404 (shown in FIG. 3) that contacts the support 120. In various embodiments, when the mounting assembly 130 is mounted onto the rail 140, turning of the fastening knob 130 may cause the fastener 404 (shown in FIG. 3) to frictionally engage the support 120. In various embodiments, variably increasing the friction between the fastener 404 and the support 120 reduces the ability of the support 120 to move relative to the mount assembly 130, including preventing the support 120 from rotating or sliding up and down in the mount assembly 130. The fastening knob 136 will be described in greater detail with reference to FIG. 3.

As previously discussed and shown in FIG. 2, in various embodiments, the mounting assembly 130 may be mounted onto the rail 140. In various embodiments, the rail 140 may include a lower side 166 and a top side 168 distal from the lower side 166. In various embodiments, the rail 140 may also include a first side 170 and a second side 172 distal from the first side 170. In various embodiments, the sides 166,168,170,172 may define a profile for the rail 140. As shown in FIG. 2, in various embodiments, the channel 152 in the adapter 132 may have a matching profile to the rail 140 such that the channel 152 may engage the rail 140 at three sides 166,168,172; however, in various other embodiments, the channel 152 may engage less than three sides of the rail 140, may engage a different combination of three sides of the rail 140, or may engage all sides 166,168,170,172.

FIG. 3 shows a cross-sectional view of the rail adapter 132 mounted on the rail 140. As previously described, the rail adapter 132 includes the lower side 144, the top side 146, the channel side 148, and the attachment side 146. In various embodiments, the channel side 148 further defines the rail mating channel 152 between the top side 146 and the lower side 144. As shown in FIG. 3, the rail mating channel 152 includes a top surface 368, a lower surface 370, and a side surface 372.

In various embodiments, the adapter 132 defines a lower bore 408 or, in various embodiments, a plurality of lower bores 408 in the lower surface 370. In various embodiments, the lower bore 408 extends through the adapter 132 from the lower surface 370 to the lower side 144. As shown in FIG. 3, in various embodiments, the lower bore 408 is threaded. In various embodiments, a lower fastener 412 is inserted into the lower bore 408. In various embodiments, the lower fastener 412 is a screw; however, in various other embodiments, the fastener may be selected from the group including, but not limited to, bolts, nuts, pins, hooks, and other suitable fasteners. In various embodiments, the lower fastener 412 is connected to or integrally formed with the securing knob 410.

In various embodiments, rotation of the securing knob 410 may cause the lower fastener 412 to contact the rail 140 and variably increase the friction between the lower fastener 412 and the rail 140. In various embodiments, rotating the securing knob 410 may cause the lower fastener 412 to contact the second engagement surface 380 or the lower side 166 of the rail 140. In various embodiments, variably increasing the degree of friction between the lower fastener 412 and rail 140 controls the freedom of movement of the mount assembly 130 along the rail 140. In various embodiments, variably increasing the friction controls the freedom of movement horizontally along the rail 140. In various other embodiments, variably increasing the friction controls the freedom of movement relative to another structure such as those structures in the group including, but not limited to, a bed, post, desk, or chair. In various embodiments, the securing knob 410 rotates and causes the lower fastener 412 to engage the rail 140 such that the mount assembly 130 is secured at one position on the structure, such as the rail 140.

In various embodiments, a ball detent (not shown) may be provided in the adapter 132. In various embodiments, the ball detent is provided in a bore adjacent to lower bore 408. In various other embodiments, there are two ball detents, each provided in a bore and spaced to either side of lower bore 408. More than two ball detents and different orientations may also be present in various other embodiments. Each ball detent is positioned in its respective bore such that the ball detent is engageable with the second engagement surface 380 or the lower side 166 of the rail 140. In various embodiments, a ball detent spring may further be included with each ball detent in its respective bore to bias the ball detent against the rail 140. In various embodiments, the bore for each ball detent may or may not pass completely through the adapter 132 and may include a plug such as a screw or bolt within each bore to hold the ball detent and the ball detent spring within the bore. Each ball detent serves to engage the adapter 132 with the rail 140 by the ball detent being held against the rail 140 in second engagement surface 380, thereby attaching the adapter 132 to the rail 140. In various embodiments, the ball detent spring holds the ball detent against the second engagement surface 380 to attach the adapter 132 to the rail 140.

In various embodiments, the rib 154 may be defined by the top surface 368. As shown in FIG. 3, the rib 154 includes a rib surface 374 and extends partially into the rail mating channel 152. In various embodiments, the rib surface 374 includes an arcuate portion; however, in various other embodiments, the rib surface may be square, angled, flat, or have any other desired shape. In various embodiments, rotating of the lower fastener 412 may further clamp or otherwise secure the rail 140 against the rib 154. In these embodiments, the rib surface 374 may provide frictional engagement with the rail 140 in addition to the frictional engagement provided by the lower fastener 412.

In various embodiments, the rail 140 defines a first engagement surface 376 in the top side 168 and a second engagement surface 380 in the lower side 166. As shown in FIG. 3, in various embodiments, the engagement surfaces 376,380 include arcuate portions which have a similar profile to the arcuate portion of the rib surface 374. In various embodiments, the presence of the engagement surfaces 376,380 allows the rail 140 to be rotated 180 degrees and serve the same function. It also allows an adapter 132 that is mounted on a rail 140 to be mounted to the left or to right of that rail 140. In the present embodiment, the adapter 132 is mounted on the rail 140 such that the rib surface 374 engages the first engagement surface 376.

As shown in FIG. 3, in various embodiments, the bore 406 of the adapter addition 134 extends through the adapter addition 134 between the lower side 158 and the top side 160 of the adapter addition 134. Additionally, as shown in FIG. 3, in various embodiments, the fastening knob 136 is connected to or integral with a fastener 404 through a threaded boss 400. In various embodiments, the threaded boss 400 enables the fastening knob 136 to engage the fastener 404; however, in various other embodiments, other mechanisms may be utilized for engaging the fastening knob 136 with the fastener 404. In various embodiments, the fastener 404 is a screw; however, in various other embodiments, the fastener 404 is selected from the group including, but not limited to, nuts, bolts, hooks, pins, and other suitable fasteners. As shown in FIG. 3, in various embodiments, the fastener 404 is inserted through a fastening opening 402 defined by the adapter addition 134. As shown in FIG. 3, in various embodiments, the fastener 404 extends through the fastening opening 402 and partially into the bore 406. In various embodiments where the support 120 is inserted into the bore 406, rotation of the fastening knob 136 may cause the fastener 404 to contact the support 120 and variably increase the friction between the fastener 404 and the support 120. In various embodiments, rotating the fastening knob 136 may cause the fastener 404 to contact the second vertical portion 362 of the support 120. In various embodiments, variably increasing the degree of friction between the fastener 404 and support 120 controls the freedom of movement of the support 120 relative to the mount assembly 130, including rotating, raising, and lowering of the support 120. In various embodiments, variably increasing the friction controls the freedom of rotation of the support 120 relative to the adapter 132 and mount assembly 130. In various embodiments, variably increasing the friction further aids in controlling the freedom of movement of the support 120 vertically relative to the adapter 132 and mount assembly 130. In various other embodiments, the freedom of movement is controlled relative to another structure such as those structures in the group including, but not limited to, a bed, post, desk, or chair. In various embodiments, the fastening knob 136 rotates and causes the fastener 404 to engage the support such that the support 120 is secured in one position.

Focusing now on FIG. 4, the hanger attachment 128, the hanger 118, and the screen 102 will be described in greater detail. As shown in FIG. 4, in various embodiments, the screen 102 may be attached to the support 120 proximate to the first end 124 of the support 120. Stated another way, the screen 102 may be attached to the support 120 nearer to the first end 124 than the second end 122 of the support 120. In various embodiments, the screen 102 may directly attach to the first end 124 of the support 120. In various other embodiments, the screen 102 may indirectly attach to the first end 124 of the support 120. For example, in various embodiments, a pivotable arm, hooks, or pins may connect the support 120 to the mount assembly 130. In other embodiments, other mechanisms for indirectly attaching the support 120 to the mount assembly 130 may be utilized.

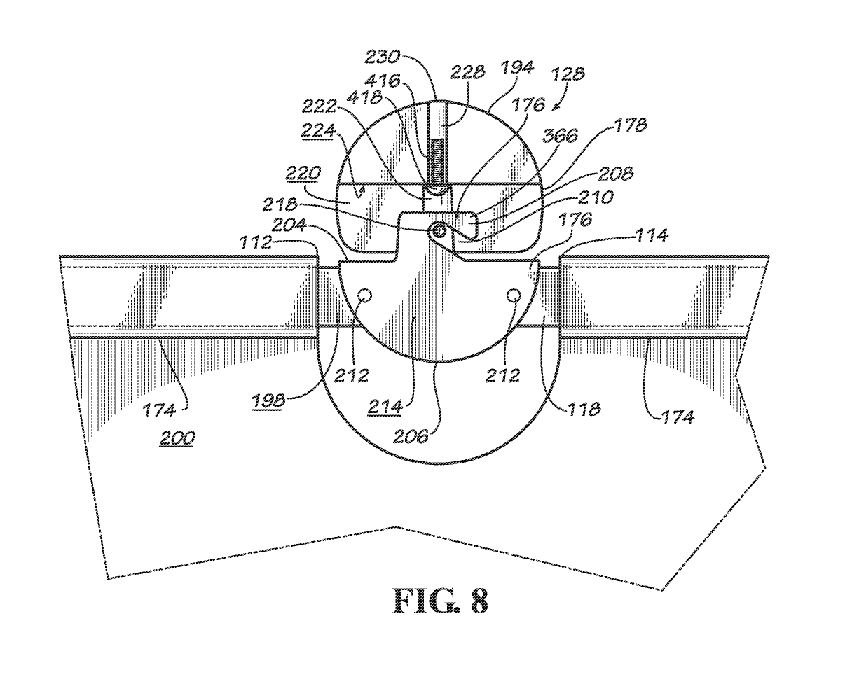

In various embodiments, the hanger attachment 128 may include a first side cap 178, a second side cap 180, and a middle insert 194 positioned between the first side cap 178 and the second side cap 180. As shown in FIG. 4, in various embodiments, the hanger attachment 128 may define a channel 182 between the first side cap 178 and the second side cap 180 and below the middle insert 194. The hanger attachment 128 will be described below in greater detail with reference to FIG. 8.

As shown in FIG. 4, in various embodiments, the screen 102 may include an opening 184 at the top end 108 of the screen 102. In the current embodiment, the opening 184 is formed by a fold in the fabric of the screen 102, though other formations of the opening 184 may be present in various embodiments. In various embodiments, the opening 184 may extend from the first end 104 to the second end 106 and may be defined between the top end 108 and a seam 174.

In various embodiments, the hanger 118 may be an elongated member including a first side 186 and a second side 188 distal from the first side 186. In various embodiments, a distance from the first side 186 to the second side 188 may define a hanger 118 width. The hanger 118 also includes a top side 192 and a lower side 190 distal from the top side 192. As shown in FIG. 4, in various embodiments, the hanger 118 may define a bar-like shape; however, in various other embodiments, the hanger 118 may have a rod shape or any other desired shape for the elongated member.

As shown in FIG. 4, in various embodiments, the hanger 118 may be inserted into the opening 184 of the screen 102. In various embodiments, the width of the hanger 118 may be less than the width of the screen 102. Additionally, as shown in FIG. 4, in various embodiments where the screen 102 includes the screen opening 116, the hanger 118 may be partially unenclosed by the opening 184 between the first end 104 and the second end 106 of the screen.

In various embodiments, the hanger 118 may include a first side screen holder 176 attached to a first side surface 198 (shown in FIG. 8) of the hanger 118 in the screen opening 116. In various embodiments, the first side surface 198 may be defined between the top side 192, the lower side 190, the first side 186, and the second side 188 of the hanger 118. In various embodiments, the hanger 118 may also include a second side screen holder 196 (shown in FIG. 5) attached to a second side surface defined between the top side 192, the lower side 190, the first side 186, and the second side 188 of the hanger 118. The first side screen holder 176 and the second side screen holder 196 will be described below in greater detail with reference to FIGS. 4, 5 and 7.

FIG. 5 shows another perspective view of the hanger 118, the hanger attachment 128, and the screen 102. As shown in FIG. 5, in various embodiments, the hanger 118 may bet attached to the first side screen holder 176 and the second side screen holder 196. Although two screen holders 176,196 are shown in the present embodiment, in various other embodiments, zero, one, two, or any other desired number of screen holders may be utilized.

As shown in FIG. 5, in various embodiments, the first side cap 178 may define a lower end 330 and a top end 336. The second side cap 180 may also define a lower end 332 and a top end 338. In various embodiments, a distance from the lower end 330 to the top end 336 of the first side cap 178 may be the same as a distance from the lower end 332 to the top end 338 of the second side cap 180. As shown in FIG. 5, the middle insert 194 may define a lower end 334 and a top end 340. In various embodiments, a distance from the lower end 334 to the top end 340 may be less than the distance from the lower end 330 to the top end 336 of the first side cap 178 or less than the distance from the lower end 332 to the top end 338 of the second side cap 180. In various embodiments, a distance from the lower end 334 to the lower end 330 of the first side cap 178 or a distance from the lower end 334 to the lower end 332 of the second side cap 180 may define the depth of the channel 182. As shown in FIG. 5, in various embodiments, the screen holders 176,196 may be inserted into the channel 182 defined by the hanger attachment 128 between the first side cap 178 and the second side cap 180 and below the middle insert 194.

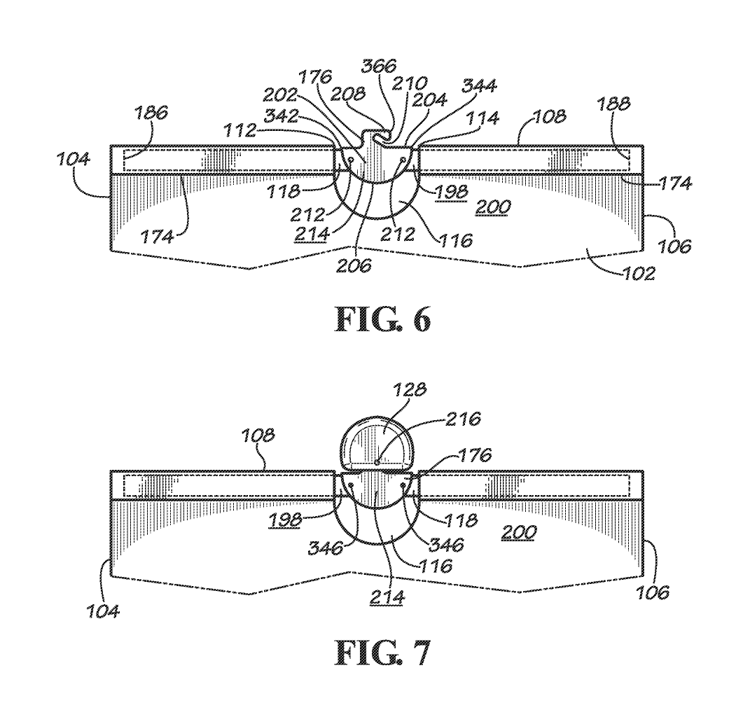

FIG. 6 shows a partial view of the screen 102 with the hanger 118 inserted. As shown in FIG. 6, in various embodiments, the first side screen holder 176 may be attached to the first side surface 198 of the hanger 118. In various embodiments, the first side screen holder 176 may define a main body 202 and an attachment portion 208. The main body 202 may include a top side 204 and a lower side 206. The main body 202 may also include a first end 342 and a second end 344 distal from the first end 342. In various embodiments, the first side screen holder may define an outer surface 214 extending between the top side 204, the lower side 206, the first end 342, and the second end 344. In various embodiments, the first side screen holder 176 may also define an inner surface extending between the top side 204, the lower side 206, the first end 342, and the second end 344. As shown in FIG. 6, in various embodiments, the lower side 206 may define a curved edge extending between the first end 342 and the second end 344; however, in various other embodiments, the lower side 206 may not define a curved edge. In various embodiments, an attachment portion 208 may extend from the top side 204 away from the main body 202. In various embodiments, the attachment portion 208 may define a hook 366; however, in various other embodiments, the attachment portion may define any other desired shape suitable for attaching the hanger 118 to the hanger attachment 128. In various embodiments where the attachment portion 208 defines a hook 366, the attachment portion 208 may define an attachment opening 210. As will be discussed below with reference to FIG. 8, in various embodiments, the attachment opening 210 may be utilized to position the first side screen holder 176 onto an attachment pin 218 (shown in FIG. 8).

As shown in FIG. 6, in various embodiments, the first side screen holder 176 may define attachment openings 212. In the present embodiment, the first side screen holder 176 includes two attachment openings 212; however, in various other embodiments, one, two, three or any other desired number of attachment openings 212 may be utilized. In various embodiments, fasteners 346 (shown in FIG. 7) may be inserted into the attachment openings 212 to secure the first side screen holder 176 to the first side surface 198 of the hanger 118. In various other embodiments, zero attachment openings 212 may be defined and the first side screen holder 176 may be attached to the first side surface 198 with fasteners coming from a group including, but not limited to, glue, double sided tape, screws, bolts, rivets, welds, and various adhesives. In various embodiments, the first side screen holder 176 may be attached to the first side surface 198 such that the inner surface of the first side screen holder 176 faces the first side surface 198; however, in various other embodiments, other configurations may be utilized. Although not shown, the second side screen holder 196 may have similar properties and dimensions as the first side screen holder 176. Additionally, although not shown, the second side screen holder 196 may be attached to the second side surface of the hanger 118 using similar mechanisms as used to attach the first side screen holder 176 to the first side surface 198.

As shown in FIG. 7, in various embodiments, the hanger 118 with the first side screen holder 176 may be engaged with the hanger attachment 128. In various embodiments, the second side screen holder 196 may also be engaged with the hanger attachment 128. In various embodiments, the attachment portion 208 of the first side screen holder 176 and the attachment portion of the second side screen holder 196 may be inserted into the hanger attachment 128 such that only the main body 202 is visible from a side view. As will be discussed below, the attachment portion 208 may engage an attachment pin 218 (shown in FIG. 8) in the hanger attachment 128. In various embodiments, the attachment pin 218 may be inserted into the pin opening 216 shown in FIG. 7. In these embodiments, the pin opening 216 may extend through the first side cap 178, the channel 182, and the second side cap 180 of the hanger attachment 128. In various other embodiments, the pin 218 may be integrally formed with the hanger attachment 128. In various other embodiments, the fastener may come from the group including, but not limited to, screws, bolts, rivets, and various adhesives to mate the hanger 118 with the hanger attachment 128.

FIG. 8 shows the hanger 118 engaged with the hanger attachment 128 with a portion of the middle insert 194 and the second side cap 180 of the hanger attachment 128 removed. As shown in FIG. 8, when the hanger 118 is engaged with the hanger attachment 128, the attachment portion 208 of the first side screen holder 174 may engage a pin 218. In various embodiments, the attachment opening 210 of the attachment portion 208 may be dimensioned to engage that pin 218 such that the hanger 118 is supported by the pin 218. As shown in FIG. 8, in various embodiments, the attachment portion 208 may be a hook 366 that engages the pin 218; however, in various other embodiments, the fasteners may come from the group including, but not limited to, screws, bolts, rivets, and various adhesives to engage the pin 218. Although not shown, in various embodiments, the attachment portion of the second side flag holder 196 may also engage the pin 218; however, in various other embodiments, a single flag holder 174 or 196 may be utilized.

As shown in FIG. 8, in various embodiments, the hanger attachment 128 may include a support bore opening 230 for a support bore 228. In various embodiments, the support bore opening 230 and the support bore 228 may be defined in the middle insert 194 of the hanger attachment 128. Although not shown, in various embodiments, the first end 124 of the support 120 may be inserted through the support bore opening 230 and into the support bore 228. In various embodiments, the first end 124 may be secured in the support bore 228 such that the hanger attachment 128 may be secured to the support. In various embodiments, the first end 124 defines a threaded bore (not shown). In various embodiments, the first end 128 may engage a fastener 416 positioned in the support bore 228. FIG. 8 shows the position of the fastener 416 in the support bore 228 in its final position after it has engaged the threaded bore of the first end 124, with the support 120 removed to show the fastener 416. In various embodiments, the fastener 416 may be selected from the group including, but not limited to, screws, nuts, bolts, pins, and other fastening mechanisms. As shown in FIG. 8, the fastener 416 includes a fastener head 418 in various embodiments with a diameter greater than a diameter of the support bore 228. In these embodiments, when the fastener 416 engages the support 120, the fastener head 418 provides a bearing surface upon which the hanger attachment 128 may pivot, swivel, or otherwise rotate relative to the support 120. In various embodiments, the first end 124 may be secured in the support bore 228 such that the hanger attachment 128 may pivot, swivel, or otherwise rotate relative to the support 120 while remaining secured to the support 120. In various embodiments, the hanger attachment 128 may be detachably connected to the support 120; however, in various other embodiments, the hanger attachment 128 may be fixably attached to the support 120.

As previously discussed, in various embodiments, the hanger attachment 128 may include the first side cap 178, the second side cap 180, and the middle insert 194. In various embodiments, the lower end 330 of the first side cap 178 may extend below the lower end 334 of the middle insert 194. In these embodiments, an inner surface 220 of the first side cap 178 may define a first side surface for the channel shown in FIG. 5. Although not shown, in various embodiments, the lower end 332 of the second side cap 180 may extend below the lower end 334 of the middle insert 194. In these embodiments, an inner surface of the second side cap 180 may define a second side surface for the channel shown in FIG. 5. In various embodiments a lower surface 224 of the middle insert 194 at the lower end 334 may define a top channel surface.

As shown in FIG. 8, in various embodiments, the inner surface 220 may define a groove 222 aligned with the support bore 228. As shown in FIG. 8, in various embodiments, a diameter of the groove 222 may be greater than the diameter of the support bore 228; however, in various other embodiments, the diameter of the groove 222 may equal the diameter of the support bore 228 or may be less than the diameter of the support bore 228. In various embodiments, the groove 222 may extend from the lower surface 224 of the middle insert 194 to the lower end 330 of the first side cap 178. Although not shown, in various embodiments, the inner surface of the second side cap 180 may also define a groove similar to the groove 222. In various embodiments, the groove 222 may be utilized to help secured the first end 124 of the support 120 in the hanger attachment 128. For example, in various embodiments, the first end 124 may include anchoring mechanisms, such as fastener 416 having the fastener head 418, which may catch in the groove 222 while allowing the hanger attachment 128 to pivot, swivel, or otherwise rotate relative to the support 120 while remaining secured to the support 120. In various other embodiments, the groove 222 may accept other mechanisms for securing the support 120 to the hanger attachment 128.

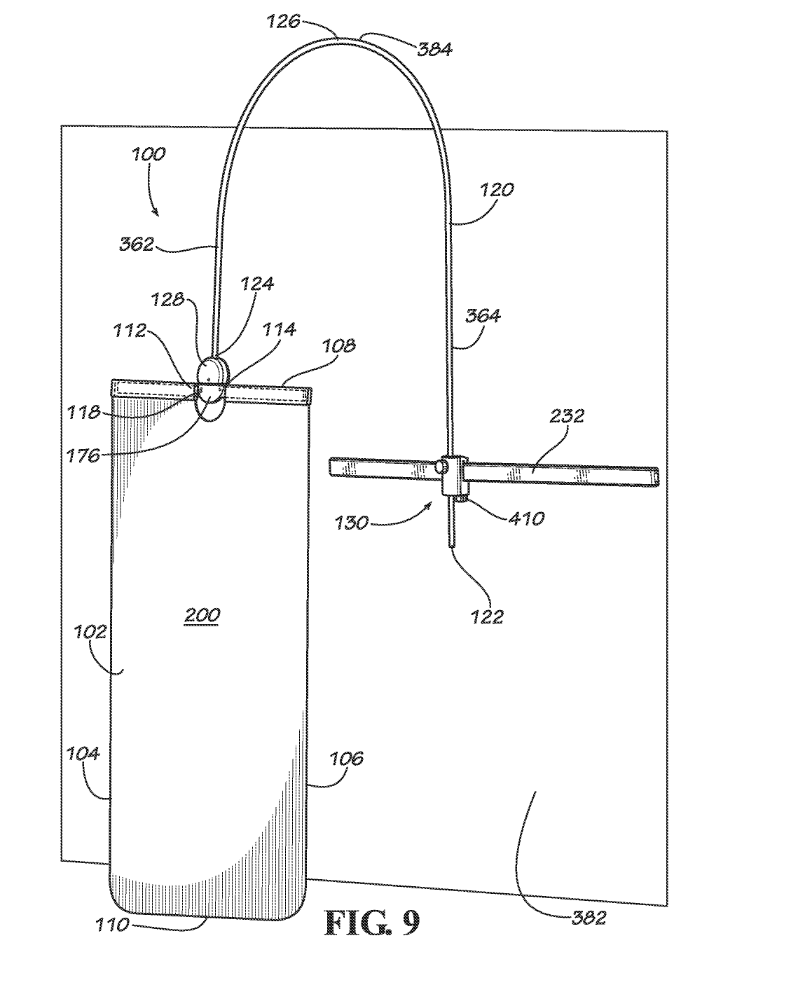

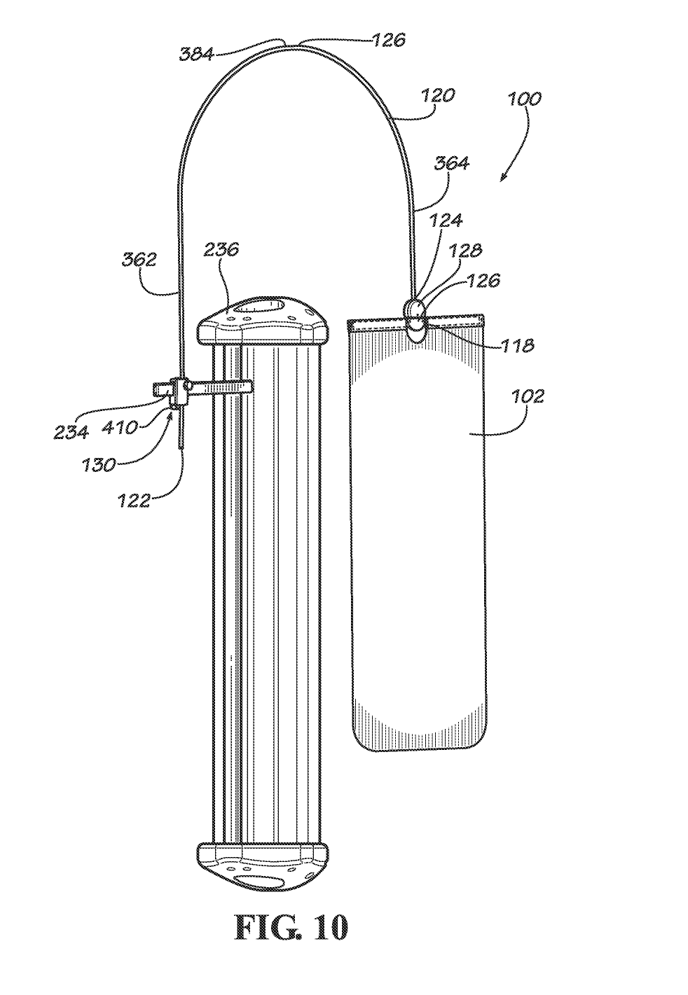

FIG. 9 shows the screen assembly 100 mounted onto a rail 232 which is mounted onto a wall 380. FIG. 10 shows the screen assembly 100 mounted onto a rail 234 which is mounted to a hospital headwall 236. For example, in various embodiments, the rail 234 may be attached to a headwall 236 such as the headwall sold under the trade name Matrixx-M™ Headwall by Nexxspan. However, in various other embodiments, the rail 234 may be attached to other types of headwalls 236. In various other embodiments, the rails 140, 232, or 234 may be any rail found within a hospital system such as a rail attached to a wall, a post, a headwall, or at any other desired location in a hospital. In these various embodiments, the rails 140, 232, or 234 may enable the screen assembly 100 to be mounted onto the rails 140, 232, or 234. In various embodiments, the rails may be mounted to other rails found within a hospital system or may not be mounted to rails at all. For example, in various embodiments, the screen assembly 100 may be directly mounted to a bed post, a chair, a desk, a wall, a bed, a cart, a door, or on various other structures, and the mount assembly 130 may be configured for connection to these structures. In various embodiments, the mount assembly 130 could be an adjustable collar for attachment to a post, a weighted stand for placement on a substantially horizontal surface, or a bracket for attachment to a substantially vertical surface, or any other suitable assembly for mounting the screen assembly 100 in place.

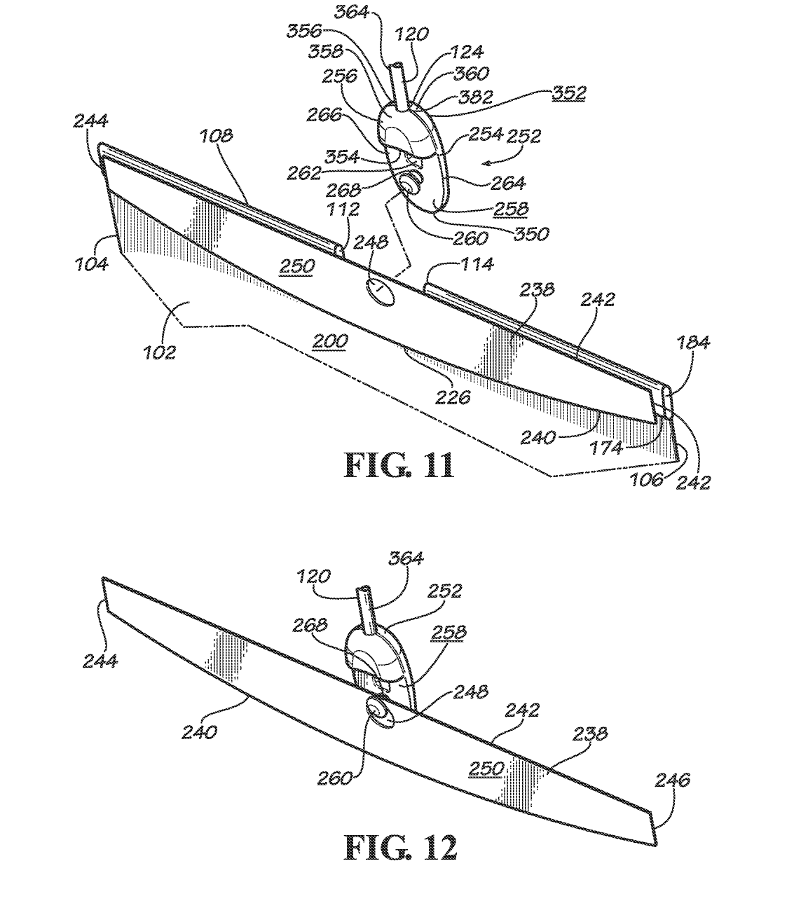

FIG. 11 shows another embodiment of a hanger attachment 252 and another embodiment of a hanger 238. Focusing first on the hanger 238, in various embodiments, the hanger 238 may include a top end 242 and a lower end 240 distal from the top end 242. The hanger 238 may also include a first end 242 and a second end 244 distal from the first end 242. As shown in FIG. 11, in various embodiments, the lower end 240 may define arcuate portion 226 between the first end 242 and the second end 244; however, in various other embodiments, the arcuate portion 226 may not be present. As shown in FIG. 11, in various embodiments, the hanger 238 may define an outer surface 250 extending between the top end 242, the lower end 240, the first end 242, and the second end 244. In various embodiments, the hanger 238 may define a hanger opening 248 through the hanger 238 extending from the outer surface 250 to the attachment surface.

As shown in FIG. 11, in various embodiments, the hanger 238 may be attached to the screen 102. In various embodiments, the hanger 238 may be attached to the screen 102 through fasteners coming from a group including, but not limited to, screws, bolts, rivets, welds, stitching and various adhesives. In various other embodiments, the hanger 238 and the screen 102 may be integrally formed such that the hanger 230 and the screen 102 constitute a single part of a screen assembly.

Focusing now on the hanger attachment 252, as shown in FIG. 11, in various embodiments, the hanger attachment 252 may include a main body 254 and a securing body 256. As shown in FIG. 11, in various embodiments, the main body 254 may include a top end 360 and a lower end 350 distal from the top end 360. In various embodiments, the main body 254 may also include a first side 264 and a second side 266 distal from the first side 264. In various embodiments, the main body 254 may define a rounded outer surface 352 extending between the top end 360, the lower end 350, the first side 264, and the second side 266. The main body 254 may also define an attachment surface 258 extending between the top end 360, the lower end 350, the first side 264, and the second side 266. As shown in FIG. 11, in various embodiments, the attachment surface 258 may be oval-shaped; however, in various other embodiments, the attachment surface 258 may have any desired shape.

In various embodiments, the main body 254 may define a fastener 260 at the attachment surface 258. In various embodiments, the fastener 260 may define an attachment groove 268. In the present embodiment, the fastener 260 is a knob having an attachment groove 268; however, in various other embodiments, a hook, a pin, or any other desired fastener 260 may be utilized. The securing body 256 of the hanger attachment 252 may be attached to the attachment surface 258 in various embodiments; however, in various other embodiments, the securing body 256 may be integrally formed with the main body 254. As shown in FIG. 11, in various embodiments, the securing body 256 may define a top end 356 and a lower end 354 distal from the top end 356. As shown in FIG. 11, in various embodiments, the lower end 350 of the main body 254 may extend lower than the lower end 354 of the securing body 256. In various embodiments, the securing body 256 may define a curved outer surface 358 similar to the curved outer surface 352 of the main body 254. In various embodiments, the securing body 256 may be attached to the main body 254 such that the top end 356 is at the same end of the hanger attachment 252 as the top end 360 of the main body 252.

In various embodiments, the securing body 256 and the main body 252 may define a support bore opening 382 and a support channel chamber 262. As shown partially in FIG. 11, the support channel chamber 262 is partially defined in the main body 252. Although not shown, in various embodiments, the first end 124 of the support 120 may be inserted through the support bore opening 382 and into the support channel chamber 262. In various embodiments, the first end 124 is secured in the support channel chamber 262 such that the hanger attachment 252 is secured to the support 120. In various embodiments, the first end 124 may be secured in the support channel chamber 262 such that the hanger attachment 252 may pivot, swivel, or otherwise rotate relative to the support 120 while remaining secured to the support 120. For example, in various embodiments, the first end 124 may include an anchoring mechanism that may catch in the support channel chamber 262 while allowing the hanger attachment 252 to pivot, swivel, or otherwise rotate relative to the support 120 while remaining secured to the support 120. In various embodiments, the first end 124 engages the fastener 416 having the fastener head 418. As described above, in various embodiments, the fastener head 418 provides a bearing surface. The bearing surface may enable the hanger attachment 252 to pivot, swivel, or otherwise rotate relative to the support 120. In various embodiments, the hanger attachment 252 may be detachably connected to the support 120; however, in various other embodiments, the hanger attachment 252 may be fixably connected to the support 120.

FIG. 12 shows the hanger 238 engaged with the hanger attachment 252. As shown in FIG. 12, in various embodiments, the fastener 260 of the hanger attachment 252 may be inserted into the hanger opening 248. In these embodiments, the hanger 238 may be supported by the hanger attachment 252 and may engage the hanger attachment 252 at the attachment groove 268.

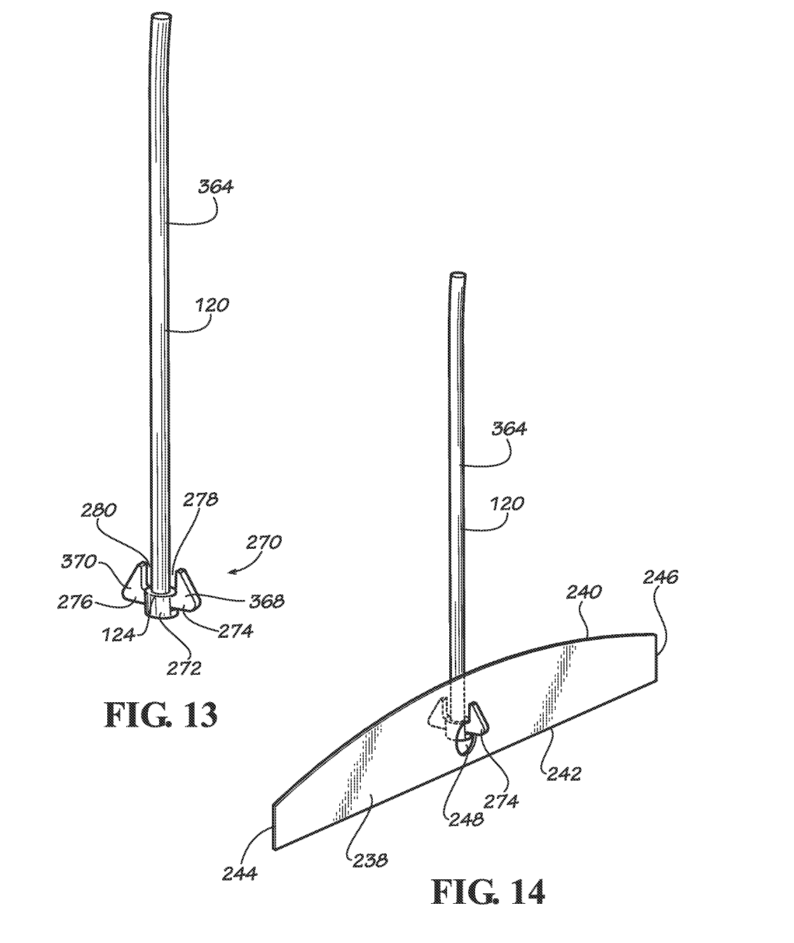

FIG. 13 shows another embodiment of a hanger attachment 270 attached to the support 120. As shown in FIG. 13, in various embodiments, the hanger attachment 270 may include a main body 272, a first fastener 274, and a second fastener 276. In various embodiments, the first end 124 of the support 120 may be inserted into an attachment opening defined in the main body 272. In various embodiments, the first end 124 may be secured to the hanger attachment 270 using methods previously described. As shown in FIG. 13, in various embodiments, the first fastener 274 may define a first attachment opening 278 and the second fastener 276 may define a second attachment opening 280. In the present embodiment, the first fastener 274 may be a first hook 368 and the second fastener 276 may be a second hook 370; however, in various other embodiments, the first fastener 274 and the second fastener 276 may be knobs, pins, or other suitable fasteners defining attachment openings 278,280.

FIG. 14 shows the hanger attachment 270 engaged with the hanger 238. As shown in FIG. 14, in various embodiments, the first fastener 274 may be inserted through the hanger opening 248 such that the hanger 238 may be supported by the hanger attachment 270 and may engage the hanger attachment 270 at the first attachment opening 278. In various other embodiments, the second fastener 276 may be inserted through the hanger opening 248 such that the hanger 238 may be supported by the hanger attachment 270 and may engage the hanger attachment 270 at the second attachment opening 280. In various other embodiments, the hanger attachment 270 may engage two hangers 238. In this embodiments, a first hanger 238 may engage the hanger attachment 270 at the first attachment opening 278 and a second hanger 238 may engage the hanger attachment 270 at the second attachment opening 280.

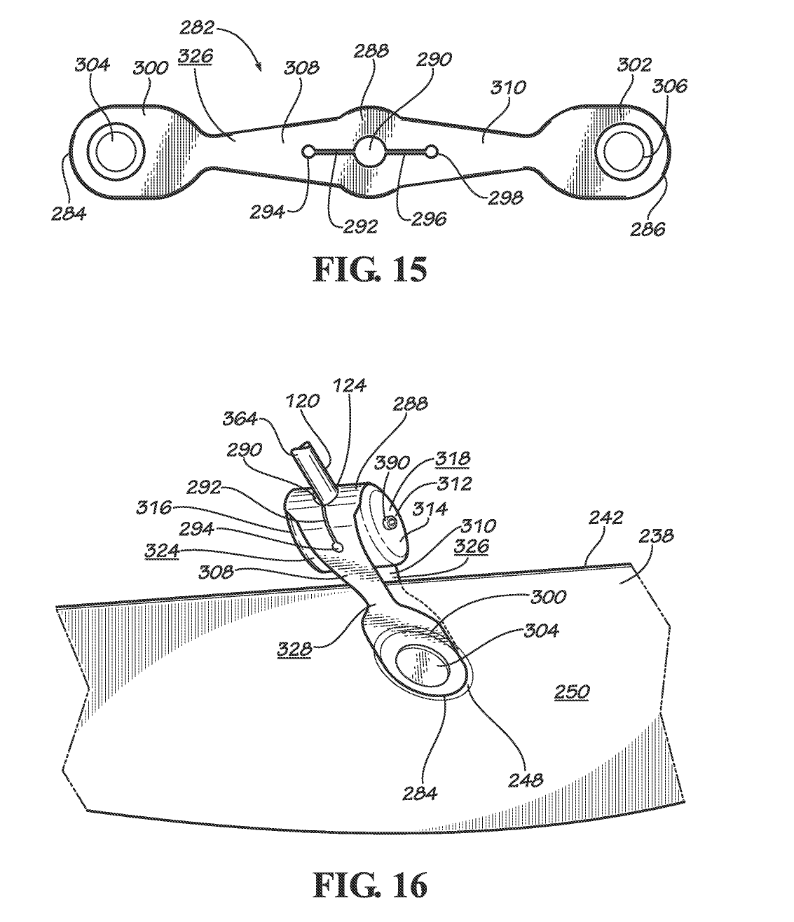

FIG. 15 shows another embodiment of a hanger attachment 282. As shown in FIG. 15, in various embodiments, the hanger attachment 282 may include a first end 284 and a second end 286 distal from the first end 284. In various embodiments, the hanger attachment 282 may define an inner surface 326 extending between the first end 284 and the second end 286. In various embodiments, the hanger attachment 282 may define an outer surface 328 (shown in FIG. 16) extending between the first end 284 and the second end 286. In various embodiments, the hanger attachment 282 is a strap or other elongated flexible member and is constructed from vinyl; however, in various other embodiments, the hanger attachment 282 may be constructed from materials in the group including, but not limited to, various plastics, textiles, and metals. These materials are flexible to enable the hanger attachment 282 to fold and bend but strong enough to support the loads placed on it supporting the hanger, as will be described below.

In various embodiments, the hanger attachment 282 may define a first body engagement portion 300 at the first end 284. As shown in FIG. 15, in various embodiments, the first body portion 300 may include a first fastener 304. In various embodiments, the hanger attachment 282 may include a second body portion 302 at the second end 286. As shown in FIG. 15, in various embodiments, the second body portion 302 may include a second fastener 306. In various embodiments, the fasteners 304,306 are magnets; however, various other fastening mechanisms may be used as the fasteners such as mechanisms from the group including, but not limited to, snaps, clasps, hooks and grooves, buttons, pins and hooks, hooks, hook and loop mechanisms (such as fasteners sold under the name VELCRO™) and other similar fastener mechanisms. In various embodiments, the hanger attachment 282 may define a center body portion 288 between the first end 284 and the second end 286. In these embodiments, the center body portion 288 may connect to the first body portion 300 through a first connecting body 308 and the center body portion 288 may connect to the second body portion 302 through a second connecting body 310.

As shown in FIG. 15, in various embodiments, the hanger attachment 282 may define a center opening 290 at the center body portion 288. In various embodiments, the hanger attachment 282 may also define a first side opening 294 in the first connecting body 308 and a second side opening 298 in the second connecting body 310. As shown in FIG. 15, in various embodiments, the first side opening 294 may connect to the center opening 290 through a first slot opening 292. In various embodiments, the second side opening 298 may connect to the center opening 290 through a second slot opening 296.

FIG. 16 shows the hanger attachment 282 engaged with the hanger 238. As shown in FIG. 16, in various embodiments where the hanger attachment 282 is utilized, a mandrel 312 may be attached proximate or near to the first end 124 of the support 120. As shown in FIG. 16, in various embodiments, the mandrel 312 may have a solid cylindrical shape; however, in various other embodiments, the mandrel 312 may have a rectangular cylindrical shape or any other suitable shape. In various embodiments, the mandrel 312 may include a first end 314 and a second end 316 distal from the first end 314. At the first end 314, the mandrel 312 may define a first side surface 318. Although not shown, in various embodiments, the mandrel 312 may also define a second side surface at the second end 316.

As shown in FIG. 16, in various embodiments, the mandrel 312 may define an outer surface 324 extending between the first end 314 and the second end 316. In various embodiments, the mandrel 312 may define a first attachment opening in the outer surface 324. In various embodiments, mandrel 312 defines a center bore extending partially through the body of the mandrel 312 and terminating at the first attachment opening of the mandrel 312. In these embodiments, the first end 124 and the first vertical portion 364 of the support 120 may be inserted into the first attachment opening and center bore to secure the mandrel 312 to the support 120. In various other embodiments, the mandrel 312 defines a second attachment opening in the outer surface 324 and distal from the first attachment opening. In these embodiments, the center bore may extend through the entire body of the mandrel 312 from the first attachment opening to the second attachment opening. In these embodiments, the first end 124 and the first vertical portion 364 can be inserted through the center bore such that the first end 124 extends below the second attachment opening in the mandrel 312 and the mandrel 312 is positioned on the first vertical portion 364 proximate to the first end 124. In various embodiments, the mandrel 312 may be retained on the support 120 using suitable retaining mechanisms such as those in the group including, but not limited to, screws, bolts, pins, washers, nuts, hooks, and other suitable securing mechanisms.

In various embodiments, the mandrel 312 further includes a set screw 390. In these embodiments, the set screw 390 is inserted into a setting opening defined in the outer surface 324 of the mandrel 312 or at end 314, the end 316, or both ends 314,316 of the mandrel 312. In the current embodiment, the setting opening is defined in end 314. In various embodiments, the mandrel 312 defines a setting bore extending from the setting opening to the center bore within the body of the mandrel 312. In various embodiments, after the set screw 390 is inserted into the setting opening and the setting bore, rotation of the set screw 390 may cause the set screw 390 to contact the first vertical portion 364 of the support 120 and variably increase the friction between the set screw 390 and the support 120. In various embodiments, variably increasing the degree of friction between the set screw 390 and support 120 controls the freedom of rotation of the mandrel 312 relative to the support 120. In various embodiments, the set screw 390 may be constructed from various plastics; however, in various other embodiments, the set screw 390 may be constructed from metals, ceramics, composites, wood, or other materials. Furthermore, in various other embodiments, setting mechanisms other than set screw 390 may be utilized to control the freedom of rotation of the mandrel 312 such as those from the group including, but not limited to, bolts, pins, nuts, hooks, and other suitable mechanisms.

As shown in FIG. 16, the hanger attachment 282 may be inserted onto the mandrel 312 such that the outer surface 324 of the mandrel 312 faces the inner surface 326 of the hanger attachment 282. As shown in FIG. 16, in various embodiments, the support 120 may extend through the center opening 290. In various embodiments, the first side opening 294, the second side opening 298, the first slot opening 292, and the second slot opening 296 may enable the hanger attachment 282 to be inserted onto the mandrel 312. In various embodiments, the hanger attachment 282 may be folded around the mandrel 312 such that the first fastener 304 engages the second fastener 306 in the hanger opening 248. In these embodiments, the inner surface 326 of the hanger attachment 282 may be adjacent to the outer surface 324 of the mandrel 312. In these embodiments, engaging the first fastener 304 with the second fastener 306 in the hanger opening 248 secures the hanger 238 to the hanger attachment 282. Where the first fastener 304 and the second fastener 306 are magnets, the magnets may be designed to be attached to each other to engage the first fastener 304 with the second fastener 306.

In various embodiments, as shown in FIG. 16, the hanger attachment 282 is first inserted onto the support 120 such that the first end 124 and first vertical portion 364 extend through the center opening 290. The mandrel 312 is then inserted onto the support 120 such that the support 120 is inserted into the center bore of the mandrel 312 and the mandrel 312 is retained on the support 120. Rotation of the mandrel 312 relative to the support 120 may be controlled by rotating set screw 390 in various embodiments. In various embodiments, after the mandrel 312 is retained, the hanger attachment 282 is positioned on the mandrel 312 such that the inner surface 326 of the hanger attachment 282 may be adjacent to the outer surface 324 of the mandrel 312. The hanger attachment 282 is then be folded around the mandrel 312 such that the first body portion 300 and the second body portion 302 hang or extend away from the mandrel 312. In various embodiments, the hanger 238 is positioned such that the fasteners 304,306 engage each other in the hanger opening 248 and secure the hanger 238 to the hanger attachment 282. Where the first fastener 304 and the second fastener 306 are magnets, the magnets may also engage each other through the hanger 238 or through screen 102 rather than through the hanger opening 248.

In various other embodiments, the hanger attachment 282 is first inserted onto the support 120 at the second end 122 before the second end 122 is received in the mounting assembly 130. In various embodiments, the mandrel 312 may be previously installed proximate to the first end 124 of the support 120 such that the hanger attachment 282 is unable to be first inserted at the first end 124. In these embodiments, the hanger attachment 282 is slid along the length of the support 120 from the second end 122 to the first end 124 until the hanger attachment 282 is positioned on the mandrel 312. The hanger 238 is then positioned such that the fasteners 304,306 engage each other in the hanger opening 248 and secure the hanger 238 to the hanger attachment 282.

In various embodiments, the hanger attachment 282 and fasteners 304,306 support the hanger 238 and screen 102. In various embodiments, the first fastener 304 remains engaged with the second fastener 306 up to specific loads being applied on the hanger 238 and screen 102. In various embodiments, the fasteners 304,306 may remain engaged in the hanger opening 248 up to a load applied on the hanger 238 and screen 102 equal to or greater than a strong pull. In these embodiments, the fasteners 304,306 detachably engaging each other provides a safety benefit of releasing upon sufficient application of force on the screen assembly 100.

A method of assembling a screen assembly 100 is also disclosed. It should be noted that any of the steps of any of the methods described herein may be performed in any order or could be performed in sub-steps that are done in any order or that are separated in time from each other by other steps or sub-steps, and the disclosure of a particular order of steps should not be considered limiting on the current disclosure.

In various embodiments, the support 120 includes the second end 122, the first end 124, the first vertical portion 364, and the second vertical portion 362. As shown in FIG. 1, in various embodiments, the hanger attachment 128 is attached proximate or near to the first end 124 of the support 120; however, in various other embodiments, the support 120 may be attached proximate or near to hanger attachment 252, hanger attachment 270, mandrel 312 and hanger attachment 282, or other suitable hanger attachments. In various embodiments, attaching the hanger attachment 128 or other suitable hanger attachments to the support 120 both secures the hanger attachment 128 to the support 120 and enables the hanger attachment to pivot, swivel, and otherwise rotate relative to the support 120.

In various embodiments, the hanger 118 may be inserted into the opening 184 of the screen 102. However, in various other embodiments, the hanger 118 and the screen 102 may be integrally formed. For example, in various embodiments, the screen 102 may be integrally formed with the hanger 118 through ultrasonic welding. In various other embodiments, other methods for integrally forming the screen 102 and the hanger 118 may be utilized. In various other embodiments, the screen 102 may be attached to the hanger 118 through stitching, adhesive, fastener, or any other suitable fastener. In various embodiments, after the hanger 118 is inserted into the screen 102, a first side screen holder 174 and a second side screen holder 196 may be attached to the sides of the hanger 118. After the screen holders 174,196 are attached to the hanger 118, the screen holders 174,196 may be inserted into the channel 182 of the hanger attachment 128 such that the hanger attachment 128 engages the screen holders 174,196. After the screen holders 174,196 are engaged, the hanger 118 and screen 102 may be supported by the support 120.

In various other embodiments, the hanger attachment 252 may be secured proximate to the first end 124 of the support 120. In these embodiments, the hanger 250 may be engaged with the fastener 260 in the hanger opening 248. In these embodiments, attaching the hanger 250 to the screen 102 may use similar methods as those described above with reference to hanger 118. In various other embodiments, the hanger attachment 272 may be secured to the first end 124 of the support. In these embodiments, the hanger 250 may be engaged with the fastener 274 in the hanger opening 248. In various other embodiments, the mandrel 312 may be secured to the first end 124 of the support 120. In these embodiments, the hanger attachment 282 may be inserted onto the mandrel 312 and may be engaged with the hanger 238 in the hanger opening 248. In various other embodiments, other suitable hanger attachments allowing the hanger 118 or 250 to detachably engage the hanger attachment may be utilized.

After the hanger attachment 128 (or hanger attachment 252, 272, or 282) engages the hanger 118 (or hanger 238), the second end 122 and second vertical portion 362 of the support 120 may be inserted through a mount assembly 130. In various embodiments, the mount assembly 130 is directly or indirectly attached proximate or near to the second end 122. In various embodiments, the second end 122 may be inserted through the adapter addition 132 attached to the adapter 132. In various embodiments, the collar 138 may be utilized to regulate how much of the second vertical portion 362 of the support 120 passes through the adapter addition 132. In various embodiments, after the second vertical portion 362 is inserted through the mount assembly 130, the support 120 may both secure the support 120 to the mount assembly 130 and offset the screen 102 from the mount assembly 130 such that the support 120 and screen 102 can pivot, swivel, and otherwise rotate relative to the mount assembly 130.

In various embodiments, the mount assembly 130 may be mounted onto a rail 140, a post, a wall, a bed post, a bed, a chair, a table, a desk, a door, or various other structures. In various embodiments where the mount assembly 130 is mounted onto a rail 14, the channel 152 of the adapter 132 may engage the rail 140 and secure the screen assembly 100 to the rail 140. In various embodiments, the rail 140 may be mounted on a post 142; however, in various other embodiments, the rail 232 may be any hospital rail or the rail 234 may be mounted to a headwall 236. In various embodiments, the mount assembly 130 may both secure the screen assembly 100 to the rail 140 (or 232 or 234) and enable the screen assembly 100 to move laterally along the rail 140. In various embodiments, the adapter 132 of the mount assembly 130 is mounted on a structure in a position to provide the best orientation for free movement of the screen assembly 100 to provide privacy for a user or to move the screen assembly 100 when privacy is no longer wanted. Fasteners of the adapter 132 such as the fastening knob 136 (or fasteners extending through the bores in the lower surface 370) may be kept in a loose configuration such that the mount assembly 130 and screen assembly 100 can be repositioned if desired. In various embodiments, if the mount assembly 130 is too loose on the structure for the user, the fastener knob 136 (or other fasteners) may be partially fastened. In various embodiments where the screen assembly 100 is mounted on a wall mounted rail, such as the rail 232, the screen assembly 100 may not be able to be rotated into a position that may disconnect the adapter 132 from the rail 232. In these embodiments, a user may choose to not fasten the fasteners.

A method of using a screen assembly 100 is also disclosed. Initially, a screen assembly 100 as described above is assembled and provided. The screen assembly 100 may be mounted onto a structure such as a rail 140, post, wall, chair, table, door, or various other structures. In the present embodiment, the screen assembly 100 is mounted on the rail 140. After the screen assembly 100 is mounted onto the rail 140, a user may laterally move the screen assembly 100 along the rail 140. After the user has selected a desired position for the screen assembly 100 along the rail 140, in various embodiments, the screen assembly 100 may be locked into that position on the rail 140. In various embodiments, the user may adjust a height of the screen assembly 100 by sliding the support 120 up or down through the mount assembly 130. In various embodiments, after a desired height is determined, the collar 138 may be positioned along the support 120 to secure the screen assembly 100 at that height.

In various embodiments, the user may then pivot, swivel, and otherwise rotate the support 120 relative to the mount assembly 130 to a desired position. In various embodiments, the user may further pivot, swivel, and otherwise rotate the screen 102 at the hanger attachment 128 relative to the support 120 to obtain a desired amount of privacy.

This assembly configuration represents one of many possible assembly configurations for the screen assembly 100. One skilled in the art will understand that obvious variations of this assembly configuration are included within this disclosure, including variations of steps, combinations of steps, and dissections of steps, among others. Where materials are chosen for the elements of this assembly, particularly rubber, metal, and plastic, similar material choices may also be used and would be obvious to one in the art. In particular, the support 120 is a rod constructed from stainless steel, injection molded plastic, or other similar materials of sufficient strength and flexibility to both support a hanger attachment 128, a hanger 118, and a screen 102 and withstand the loads placed on it while moving the screen assembly 100 and attaching and detaching the hanger 118 and screen 102. The hanger 118 (or hanger 250) may be constructed from various plastics, corrugated cardboard, metals, or any other material of sufficient strength to support a screen 102 but flexible and lightweight to reduce the weight supported by the support 120. The screen 102 may be constructed from flexible materials such as plastics, resins, textiles, and other similar materials that may be both pleasing to the touch but promote a safe medical environment. In various embodiments, the screen 102 may be constructed from nonwoven tough polyester. In particular, in various embodiments, the screen may be constructed from a nonwoven tough polyester such as the material sold by E.I. du Pont de Nemours and Company (DuPont), Wilmington, Del. under the trade name TYVEK®, or any other similar material. In various embodiments, the material chosen for the screen 102 may be a material that a user can write on. In various embodiments, materials such as felt tipped pens, markers, or other similar materials may be used by a user to write on the screen. In various embodiments, writing on the screen may serve utilitarian purposes such as recording medical considerations. In other embodiments, writing on the screen may serve aesthetic purposes such as a personalized screen with images and words of support. In various other embodiments, images, words, and other designs may be printed on the screen 102 prior to use. Finally, additional members may be added to the screen assembly 100, various components may be split into other components, and various components may be combined into single components. For one example among others, the hanger 118 and the screen 102 may be integrally formed using molding technology, ultrasonic welding, or other methods known in the art. For another example among others, the adapter 132 and adapter addition 132 may be integrally formed as a single component.

These together with other objects of the invention, along with various features of novelty that characterize the invention, are pointed out with particularity in the further description annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

One should note that conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment.

It should be emphasized that the above-described embodiments are merely possible examples of implementations, merely set forth for a clear understanding of the principles of the present disclosure. Any process descriptions or blocks in flow diagrams should be understood as representing modules, segments, or portions of code which include one or more executable instructions for implementing specific logical functions or steps in the process, and alternate implementations are included in which functions may not be included or executed at all, may be executed out of order from that shown or discussed, including substantially concurrently or in reverse order, depending on the functionality involved, as would be understood by those reasonably skilled in the art of the present disclosure. Many variations and modifications may be made to the above-described embodiment(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

That which is claimed is:

1. A privacy screen assembly comprising:
a screen;
a support having a first end and a second end, the screen attached to the support proximate to the first end; and
a mount assembly, the mount assembly attached to the support proximate to the second end, the support offsetting the screen from the mount assembly.

2. The screen assembly of claim 1, wherein the support is pivotable relative to the mount assembly and the screen is pivotable relative to the first end of the support.

3. The screen assembly of claim 1, further comprising a hanger attachment, the hanger attachment attached to the first end of the support, the hanger attachment attaching the support to the screen with a detachable connection.

4. The screen assembly of claim 3, further comprising a hanger attached to the screen, wherein the hanger attachment and the hanger define the detachable connection with the screen.

5. The screen assembly of claim 4, wherein the detachable connection includes a hanger opening defined on the hanger, the hanger opening sized to accept a portion of the hanger attachment.

6. The screen assembly of claim 3, wherein the hanger attachment includes at least one of:
a pin and groove;
a knob;
a hook; and
a first magnet and a second magnet on an elongated flexible member.

7. The screen assembly of claim 1, further comprising a screen holder attached to the screen, the screen holder attaching the screen to the support with a detachable connection.

8. The screen assembly of claim 7, wherein the screen holder includes a hook, the hook attaching the screen to the support.

9. The screen assembly of claim 1, wherein the support is a rod having a vertical portion and an arcuate portion.

10. The screen assembly of claim 1, wherein the mount assembly is adapted to attach to a rail.

11. A method of assembling a privacy screen assembly comprising:
attaching a screen proximate to a first end of a support; and
attaching the support to a mount assembly proximate to a second end of the support such that the support offsets the screen from the mount assembly.

12. The method of claim 11, wherein:
a hanger attachment is attached to the first end of the support;
the screen is attached to a hanger; and
attaching the screen proximate the first end of the support includes attaching the hanger with the hanger attachment.

13. The method of claim 12, wherein the hanger attachment is detachable from the hanger.

14. The method of claim 12, wherein the hanger attachment includes a first magnet and a second magnet on an elongated flexible member, the elongated flexible member folded around a mandrel attached to the first end of the support, and wherein attaching the hanger with the hanger attachment includes engaging the first magnet with the second magnet.

15. The method of claim 11, further comprising mounting the mount assembly on a rail.

16. A method of using a screen assembly comprising:
mounting the screen assembly onto a structure, the screen assembly including:
a screen;
a support having a first end and a second end, the screen attached to the support proximate to the first end; and
a mount assembly, the mount assembly attached to the support proximate to the second end, the support offsetting the screen from the mount assembly; and
moving the screen relative to the mount assembly.

17. The method of claim 16, wherein mounting the screen assembly onto the structure includes attaching the mount assembly to a rail.

18. The method of claim 16, wherein moving the screen relative to the mount assembly includes rotating the support.

19. The method of claim 16, wherein the screen assembly includes a hanger and a hanger attachment, the hanger attachment attached to the first end of the support and the hanger attached to the screen, and wherein the screen is attached to the support by engagement of the hanger with the hanger attachment.

20. The method of claim 16, further comprising detaching the screen from the support.
